SEQUENCE LISTING (33 sequences)

SEQ ID NO: 1 - DNA, 59 bp, Artificial Sequence
  Synthesized
atacacaagttgtggtgcaagagaccgaattcggtctcagttttagagctagaaatagc

SEQ ID NO: 2 - DNA, 41 bp, Artificial Sequence
  Synthesized
atgggttagttgtggcaaaaaaagcaccgactcggtgccac

SEQ ID NO: 3 - DNA, 20 bp, Artificial Sequence
  Synthesized
gccacaactaacccatctcc

SEQ ID NO: 4 - DNA, 22 bp, Artificial Sequence
  Synthesized
ccacaacttgtgtatcccattg

SEQ ID NO: 5 - DNA, 42 bp, Artificial Sequence
  Synthesized
atgggatacacaagttgtggggtgcttgatgctttggataag

SEQ ID NO: 6 - DNA, 44 bp, Artificial Sequence
  Synthesized
ccgaattcggtctcttgcaaccacagtaagtacttgtagttaag

SEQ ID NO: 7 - DNA, 21 bp, Artificial Sequence
  Synthesized
tgcaagagaccgaattcggtc

SEQ ID NO: 8 - DNA, 22 bp, Artificial Sequence
  Synthesized
ccacaacttgtgtatcccattg

SEQ ID NO: 9 - DNA, 21 bp, Artificial Sequence
  Synthesized
agagaccgaattcggtctcag

SEQ ID NO: 10 - DNA, 22 bp, Artificial Sequence
  Synthesized
acatcaggacctagagttcacc

SEQ ID NO: 11 - DNA, 32 bp, Artificial Sequence
  Synthesized
gactccatggttttagagctagaaatagcaag

SEQ ID NO: 12 - DNA, 30 bp, Artificial Sequence
  Synthesized
gctactaccaaacatcaggacctagagttc

SEQ ID NO: 13 - DNA, 50 bp, Artificial Sequence
  Synthesized
aaaaaaaaaaaaatgtttgatcagatcattcaaatctgatggtgcccatc

SEQ ID NO: 14 - DNA, 41 bp, Artificial Sequence
  Synthesized
ttacttagaaacggaagaagaatcatcacatccaacagaat

SEQ ID NO: 15 - DNA, 45 bp, Artificial Sequence
  Synthesized
tcttcttccgtttctaagtaaggtgcttgatgctttggataaggc

SEQ ID NO: 16 - DNA, 54 bp, Artificial Sequence
  Synthesized
gaatgatctgatcaaacatttttttttttttaaaaaaagcaccgactcggtgcc

SEQ ID NO: 17 - DNA, 44 bp, Artificial Sequence
  Synthesized
gactccatggttttagagctagaaatagcaagttaaaataaggc

SEQ ID NO: 18 - DNA, 43 bp, Artificial Sequence
  Synthesized
gctactaccaattacttagaaacggaagaagaatcatcacatc

SEQ ID NO: 19 - DNA, 35 bp, Artificial Sequence
  Synthesized
gttttagagctagaaatagcaagttaaaataaggc

SEQ ID NO: 20 - DNA, 33 bp, Artificial Sequence
  Synthesized
ttacttagaaacggaagaagaatcatcacatcc

SEQ ID NO: 21 - DNA, 861 bp, Artificial Sequence
  Synthesized
ggatgtgatgattcttcttccgtttctaagtaacgaagagcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagctcttcggttttagagctagaaatagc

SEQ ID NO: 22 - DNA, 50 bp, Artificial Sequence; identical to SEQ ID NO: 13
  Synthesized

SEQ ID NO: 23 - DNA, 47 bp, Artificial Sequence
  Synthesized
ttcttcttccgtttctaagtaacgaagagcatgggggaagcggtgat

SEQ ID NO: 24 - DNA, 23 bp, Artificial Sequence
  Synthesized
taaggtgaagcagaagcttaagc

SEQ ID NO: 25 - DNA, 23 bp, Artificial Sequence
  Synthesized
aacgcttaagcttctgcttcacc

SEQ ID NO: 26 - DNA, 24 bp, Artificial Sequence
  Synthesized
tcaagagacttgcgtcatcttccc

SEQ ID NO: 27 - DNA, 26 bp, Artificial Sequence
  Synthesized
gcatgctcaactgaaattgagtcgtc

SEQ ID NO: 28 - DNA, 23 bp, Artificial Sequence
  Synthesized
taattgttgccgtaggtgcccgg

SEQ ID NO: 29 - DNA, 23 bp, Artificial Sequence
  Synthesized
aacccgggcacctacggcaacaa

SEQ ID NO: 30 - DNA, 22 bp, Artificial Sequence
  Synthesized
ccttcatccttcagccatgcat

SEQ ID NO: 31 - DNA, 23 bp, Artificial Sequence
  Synthesized
ccactaaatgcctatcacatacg

SEQ ID NO: 32 - DNA, 23 bp, Artificial Sequence
  Synthesized
agggcaattcacatgccactgat

SEQ ID NO: 33 - DNA, 25 bp, Artificial Sequence
  Synthesized
cctccatttttccacatcttagtcc

(19) United States
(12) Patent Application Publication
(10) Pub. No.: US 2021/0348176 A1
(43) Pub. Date: Nov. 11, 2021

(54) BARLEY STRIPE MOSAIC VIRUS-BASED GENE EDITING VECTOR SYSTEM

(71) Applicant: China Agricultural University, Beijing (CN)

(72) Inventors: Yongliang Zhang, Beijing (CN); Jiacheng Hu, Beijing (CN); Dawei Li, Beijing (CN); Zhihao Jiang, Beijing (CN); Zhaolei Li, Beijing (CN)

(21) Appl. No.: 17/235,432

(22) Filed: Apr. 20, 2021

Related U.S. Application Data
(63) Continuation-in-part of application No. PCT/CN2019/113013, filed on Oct. 24, 2019.

(30) Foreign Application Priority Data
Oct. 24, 2018 (CN) 2018112434739

(51) Int. Cl. C12N 15/82; C12N 15/113; C12N 15/86

(52) U.S. Cl. CPC C12N 15/8203; C12N 15/113; C12N 2770/00043; C12N 2310/12; C12N 15/86

(57) ABSTRACT

A Barley stripe mosaic virus-based gene editing vector system, comprising artificial plasmids separately containing Barley stripe mosaic virus RNAα, RNAβ, and RNAγ. The required sgRNA sequence is integrated in RNAβ or RNAγ. The Barley stripe mosaic virus-based gene editing vector system can perform efficient gene editing on genomes of dicotyledons such as Nicotiana benthamiana and monocotyledons such as wheat and maize. Using the gene editing vector system, users can directly obtain wheat seeds harboring targeted gene editing events simply by inoculating the Cas9-transgenic wheat, and the edited target gene is transmitted to progeny plants through the seeds, without the need for transformation, tissue culture and regeneration process

### CROSS REFERENCE

This application is a continuation-in-part of International Application No. PCT/CN2019/113013, filed Oct. 24, 2019, and entitled “Barley Stripe Mosaic Virus-based Gene Editing Vector System,” which claims priority to Chinese Application 2018112434739, filed Oct. 24, 2018, of each of which are hereby incorporated by reference in their entirety.

### TECHNICAL FIELD

The present invention belongs to the field of biotechnology, and specifically relates to a barley stripe mosaic virus-based gene editing vector system.

### BACKGROUND ART

The gene editing efficiency of the CRISPR/Cas9 technology is closely correlated with the accumulation levels of the Cas9 protein and sgRNA in target cells. Generally, the gene editing vector based on a transient expression vector can be introduced into plant tissues through biolistic bombardment, or introduced into plants through Agrobacterium-mediated transformation. However, since the transient expression vector itself cannot replicate and transfer into the target cells, the number of cells that can finally obtain the sgRNA and Cas9 protein is limited. For the cells that obtained the Cas9 protein and sgRNA, the accumulation levels of both are also limited, thereby adversely affecting the efficiency of gene editing. For this reason, it is often necessary to transform a large number of recipient tissues at the same time, and the target mutants can be possibly screened out by performing a large-scale tissue culture operation, which is time-consuming, laborious, and costly. In contrast, the plant virus-mediated gene editing vectors (referred as “VMGE vectors” hereafter) can replicate efficiently in the host plant cells, and thus the accumulation levels of the sgRNA and/or Cas9 protein in target cells can be effectively improved. In addition, some VMGE vectors can still retain the ability of systemic movement in host plants, so that the proportion of cells that can obtain the sgRNA and/or Cas9 protein is greatly increased, which is beneficial to improve the editing efficiency of target genes. Meanwhile, the VMGE vectors are also relatively simple to operate, which can infect the host plants by Agrobacterium-mediated infiltration or rub-inoculation, and achieving targeted gene editing of endogenous genes of the host plants.

The currently reported VMGE vectors mainly include plant geminivirus, Tobacco rattle virus (TRV), Tobacco mosaic virus (TMV), each of which has some important advantages as well as some constraints.

Plant geminiviruses are DNA viruses. After infecting and proliferating within the plant cells, geminiviruses may compete the replication and translation machineries with the host cells, which may have deleterious effect on the normal growth of plants, and tissues infected by geminiviruses are difficult to regenerate. Geminivirus replicon-based VMGE vectors are often unable to move and spread in plants. In addition, the geminivirus is still one of the most threatening viruses for crops. It can cause severe symptoms and can lead to huge damage to the crops. Furthermore, the geminivirus can be transmitted by insects (such as Bemisia tabaci), which is difficult to control.

In 2016, an RNA virus-based VMGE vector was reported, the TRV-based VMGE vector. The TRV genome contains two single-stranded RNAs. Although the TRV-based gene editing vector maintains the ability of systemic movement, the host range of TRV is relatively limited, and it generally does not infect monocotyledons including important crops such as barley, wheat, and maize, thus limiting its application to these important crops.

In 2017, a TMV-based VMGE vector was reported. However, the movement protein in this vector was removed, thus losing the ability of systemic movement, which limits its usage only in the inoculated leaves. In addition, the TMV also cannot infect gramineous crops such as wheat and maize.

Therefore, there is an urgent need to develop VMGE vector that can be applied to monocotyledonous gramineous crops (such as wheat), and retain the systemic movement ability and replication ability of the virus in the host plant.

### SUMMARY OF THE INVENTION

In order to solve the problems in the prior art, the purpose of the present invention is to provide a Barley stripe mosaic virus (BSMV)-based gene editing vector system.

In order to achieve the purpose of the present invention, the technical solutions of the present invention are as follows:

In a first aspect, the present invention provides a BSMV-based gene editing vector system.

The BSMV is a multipartite RNA virus, the genome of which contains three single-stranded positive genomic RNAs, which are called RNAα (GenBank: U35767.1), RNAβ (GenBank: U35770.1) and RNAγ (GenBank: U13917.1), respectively.

Wherein, the aa protein encoded by the RNAα and the γa protein encoded by the RNAγ constitute the virus replicase; the RNAβ encodes the viral coat protein CP and the triple gene block movement complexes (TGBs); the RNAγ also encodes a small protein γb, which is a multifunctional protein. When RNAα, RNAβ, and RNAγ coexist, the virus can replicate and move in the host plant; and co-inoculation of RNAα and RNAγ is sufficient for the virus to replicate in the host plant. The BSMV can infect a variety of monocotyledonous plants including barley, wheat, maize, millet, etc. and the dicotyledon Nicotiana benthamiana.

It should be noted that when the gene editing vector system needs to be applied to gene editing of maize, the RNAβ is a β chain that has been mutated on the basis of the wild-type BSMV RNAβ (GenBank: U35770.1). The mutation causes the amino acid G (glycine) to be mutated to E (glutamic acid) at position 404 of the TGB1 protein encoded by the RNAβ. For more detailed information, please refer to Hu Yue's doctoral dissertation (Functional analysis of the Barley stripe mosaic virus TGB1 protein phosphorylation in viral infection and movement, Hu Yue, doctoral dissertation, China Agricultural University, 2015).

The BSMV-based VMGE vector system provided by the present invention comprises artificial plasmids containing the above-mentioned RNAα, RNAβ, and RNAγ, respectively; and a required sgRNA sequence is integrated into the RNAβ or RNAγ.

That is, the vector system comprises: (1) an artificial plasmid containing RNAα, (2) an artificial plasmid containing RNAβ and integrated with sgRNA, and (3) an artificial plasmid containing RNAγ; or

the vector system comprises: (1) an artificial plasmid containing RNAα, (2) an artificial plasmid containing RNAβ, and (3) an artificial plasmid containing RNAγ and integrated with sgRNA.

The artificial plasmid is preferably a plasmid containing an HDVRz ribozyme. The HDVRz ribozyme was used to correctly transcribe the 3′ untranslated region of the genomic RNAs of BSMV, thus ensuring the infectivity of BSMV in the host plants.

The artificial plasmids containing the HDVRz ribozyme include, but are not limited to pCB301, pCass4-Rz.

In a specific embodiment of the present invention, the artificial plasmid pCB301 is used to construct the vector system. The artificial plasmid pCB301 is provided by professor Tao Xiaorong of Nanjing Agricultural University (Yao, M., Zhang, T., Tian, Z., Wang, Y, Tao, X., 2011. Construction of Agrobacterium-mediated cucumber mosaic virus infectious cDNA clones and 2b deletion viral vector. Scientia Agricultura Sinica, 2011, 44 (26): 4886-4890.)

The artificial plasmid pCB301 contains a multiple cloning site, and three genomic RNAs of the BSMV are cloned into the pCB301 through two restriction sites of StuI and BamHI therein, and the products are called pCB301-BSMVα, pCB301-BSMVβ and pCB301-BSMVγ, respectively.

It has been discovered through experimental research in the present invention that there are multiple sites on the BSMV genome where exogenous fragments can be inserted without affecting the replication and movement ability of the virus itself, such as the 5′ end and 3′ end of γb; and the middle part of the coding sequence of the coat protein CP can be substituted with exogenous fragments without affecting the replication and movement ability of the virus.

Therefore, when the required sgRNA sequence is integrated into the RNAβ, in order to prevent the integrated sgRNA sequence from affecting the systemic movement of the virus, the sgRNA expression cassette and the upstream and downstream sequences are subjected to insert in the region between 74 nt and 435 nt of the nucleoside sequence of the CP gene (the gene encoding the CP protein). In a specific embodiment of the present invention, as an exemplary operation, the sgRNA expression cassette together with the upstream and downstream sequences are substituted for the 74-393 nucleotide sequence of the CP gene.

When the required sgRNA sequence is integrated into the RNAγ, in order to prevent the integrated sgRNA sequence from affecting the systemic movement of the virus, the present invention chooses to insert the required sgRNA at the 3′ end of the γb ORF.

A person skilled in the art should understand that sgRNA comprises two parts, i.e., the spacer part at the 5′ end and a closely linked scaffold part at the 3′ end. The nucleotide sequence of the spacer can be changed, and its length can also vary according to the Cas9 protein used. In the technical solution of the present invention, the upstream of the spacer and/or the downstream of the scaffold part allow for additional sequences in addition to the sequence of the genome of the BSMV itself, which may be the sequences of another or multiple sgRNAs and/or other sequences other than the virus itself and the sgRNA.

Furthermore, the present invention specifically describes the sgRNA sequence integration involved in the technical solution as follows:

In the present invention, the required sgRNA sequence is obtained by PCR amplification or synthesis, and the sgRNA sequence is cloned to the above-mentioned specific position by methods such as ligation after endonuclease digestion or homologous recombination cloning.

Specifically, the viral vector is first linearized by inverse PCR, the sgRNA expression cassette is amplified or synthesized, and about 20 bp of a sequence homologous to the viral vector is added at both ends, then the sgRNA is cloned onto the viral vector through a recombination reaction, and the insertion site thereof can be inside the CP or after the γb. Additional sequences can also be added to both ends of the sgRNA sequence. The so-called additional sequence can be a sequence on the viral vector, a sequence on the sgRNA, or any other sequence, and its length can be ranged from several bp to several hundred bp. Of course, the sgRNA sequence can also be cloned into the viral vector by methods such as ligation after endonuclease digestion.

In a specific embodiment of the present invention, the sgRNA is designed to target the Nicotiana benthamiana PDS gene, and the sgRNA is integrated into CP. The gene editing vector system comprises: (1) pCB301 containing RNAα, (2) pCB301 containing RNAβ and integrated with sgRNA, and (3) pCB301 containing RNAγ.

In another specific embodiment of the present invention, the sgRNA designed to target the Nicotiana benthamiana PDS gene is integrated downstream the γb gene, and the gene editing vector system comprises: (1) pCB301 containing RNAα, (2) pCB301 containing RNAβ, and (3) pCB301 containing RNAγ and integrated with sgRNA.

The Barley stripe mosaic virus-based gene editing vector system of the present invention can perform efficient gene editing on the genomes of dicotyledons such as Nicotiana benthamiana and monocotyledons such as wheat and maize. The gene editing vector system of the present invention can directly obtain wheat seeds containing edited target gene by inoculating the Cas9 transgenic wheat, and the edited target gene is transmitted to progeny plants through the seeds, without the need for transformation, tissue culture and regeneration processes.

In a second aspect, the present invention provides application instances of the gene editing vector system in plant gene editing.

In the present invention, the plants are monocotyledons or dicotyledons.

The monocotyledons include, but are not limited to barley, wheat, oat, maize, and millet. The dicotyledons include, but are not limited to Nicotiana benthamiana.

Furthermore, the gene editing vector system of the present invention can realize gene editing in monocotyledons, and overcomes the defects and deficiencies of the prior art that the TRV or TMV-based VMGE vectors cannot infect gramineous crops such as wheat and maize.

In a third aspect, the present invention also provides a method for gene editing of plants, which is performed by using the VMGE vector system of the present invention.

The VMGE vector system of the present invention can be introduced into plant cells by methods such as biolistic bombardment, rub-inoculation with in vitro transcription products, and Agrobacterium-mediated infiltration, and can replicate and systemically move in plants such as barley, wheat, maize, Brachypodium sylvaticum, Chenopodium Amaranticolor, and Nicotiana benthamiana. The BSMV-based VMGE vector contains a sgRNA sequence, and the editing of genomes of plants such as Nicotiana benthamiana can be achieved in the presence of Cas9 protein. For example, the BSMV-based gene editing vector is inoculated onto Nicotiana benthamiana by Agrobacterium-mediated infiltration, and targeted gene editing can be achieved both in the inoculated area and systemically infected plant tissues of the BSMV-based VMGE vector in the presence of the Cas9 protein.

It has been proved through experiments in the present invention that Nicotiana benthamiana infected by the BSMV-based VMGE vector of the present invention by Agrobacterium-mediated infiltration, targeted gene editing can be achieved both in the inoculated area and systemically infected plant tissues of the BSMV-based VMGE vector in the presence of the Cas9 protein, with an efficiency more than 70%.

Furthermore, the BSMV-based VMGE vector of the present invention enables editing on the endogenous genes of systemically infected leaves of wheat and maize in the presence of the Cas9 protein with in vitro transcripts of the BSMV-based VMGE vector by rub-inoculation.

The beneficial effects of the present invention lie in that:

The present invention provides a BSMV-based VMGE vector system, wherein a part of the CP sequence can be substituted with the sgRNA sequence, or the sgRNA can be cloned to the 3′ end of γb ORF without affecting its infectivity of BSMV on the host plants. Additional nucleotide sequences can be added to both ends of the sgRNA sequence without disrupting its normal function. The BSMV-based VMGE vector can achieve targeted gene editing in the plant genome beyond the inoculated area due to the movement ability. Importantly, targeted gene editing can be achieved in the offspring of the BSMV-based VMGE vector infected Cas9-transgenic wheat plants though seeds, which can circumvent the difficult transformation and regeneration processes.

Compared with the previously reported TRV-based VMGE vectors, the vector system of the present invention can infect some important crop species, including barley, wheat, oat, millet and maize.

Compared with the previously reported TMV-based VMGE vector, the vector system of the present invention retains the systemic movement ability and replication ability of the virus in the host plants, thereby enabling gene editing of a larger range within the plants, not just limited to the inoculated areas.

Compared with the previously reported geminiviruses-based VMGE vectors, the vector system of the present invention, under the premise of achieving a high editing efficiency, it will not compete with plant cells for factors necessary for replication and translation and will not interfere with the cell cycle of the plants. In addition, the BSMV, as an RNA virus, will not integrate into the plant genome, which may introduce additional exogenous fragments into the plant genome. Moreover, sgRNA can be integrate into different genomic RNAs of the BSMV-based VMGE vector provided by the present invention.

### BRIEF DESCRIPTION OF THE DRAWINGS

FIG. 1 is a schematic diagram of the β-CP-Tgcas-gNbPDS4 vector described in Example 1.

FIG. 2 is a schematic diagram of the γ-gRNA-gNbPDS4 vector described in Example 2.

FIG. 3 is a schematic diagram of the cDNA structure of BSMV RNAγ contained in the pT7-ge-BSγ-SmR-TaGASR7-T1 in Example 3.

FIG. 4 is a diagram showing the result of enzymatic digestion of the NbPDS544 fragment in Experimental Example 1.

FIG. 5 is a diagram showing the sequencing results of the NbPDS544 fragment cloned into the T-vector in Experimental Example 1.

FIG. 6 is a diagram showing the result of enzymatic digestion of the NbPDS544 fragment in Experimental Example 1.

FIG. 7 is a diagram showing the sequencing result of the NbPDS544 fragment cloned into the T-vector in Experimental Example 1.

FIG. 8 is a diagram showing the results of enzymatic digestion of the TaGASR7-A1, TaGASR7-B1, and TaGASR7-D1 fragments in Experimental Example 2.

FIG. 9 is a diagram showing the sequencing results of the TaGASR7-A1, TaGASR7-B1, and TaGASR7-D1 fragments cloned into the T-vector in Experimental Example 2.

FIG. 10 is a diagram showing the results of enzymatic digestion of the ZmTMS5-994 fragment in Experimental Example 2.

FIG. 11 is a diagram showing the sequencing results of the ZmTMS5-994 cloned into the T-vector in Experimental Example 2.

FIG. 12 is a diagram showing the results of enzymatic digestion of the TaGASR7-A1, TaGASR7-B1, and TaGASR7-D1 fragments and the corresponding sequencing results in Experimental Example 3.

FIG. 13 is a diagram showing the results of enzymatic digestion of the TaGASR7-A1, TaGASR7-B1, and TaGASR7-D1 fragments in Experimental Example 3.

FIG. 14 is a diagram showing the sequencing results of the TaGASR7-A1, TaGASR7-B1, and TaGASR7-D1 fragments that cloned into the T-vector in Experimental Example 3.

### SPECIFIC MODES FOR CARRYING OUT THE EMBODIMENTS

In the following, the present invention will be further explained in conjunction with Examples. It should be understood that the following Examples are given for illustrative purposes only, and are not intended to limit the scope of the present invention. A person skilled in the art can make various modifications and substitutions to the present invention without departing from the purpose and spirit of the present invention.

The experimental methods used in the following Examples are conventional methods unless otherwise specified.

The materials and reagents used in the following Examples can be obtained from commercial sources unless otherwise specified.

### Example 1

In this Example, the Nicotiana benthamiana PDS (NbPDS) gene was used as a target gene to illustrate the construction and application of a Barley stripe mosaic virus-based gene editing vector system.

I. Construction

1. Three genomic RNAs of the Barley stripe mosaic virus were cloned into pCB301 vector through the StuI and BamHI restriction sites, and the products were called pCB301-BSMVα, pCB301-BSMVβ and pCB301-BSMVγ, respectively.

2. The sgRNA sequence contains at least two parts, the first part is the so-called spacer part at the 5′ end of the sequence, the length of which is about 20 bp, and the other part is the so-called sgRNA scaffold part. The scaffold part was synthesized and cloned into the pENTR4-gRNA7 vector by GENEWIZ Company.

3. Primers F1 and R1 were designed and synthesized by Invitrogen Company. The sequences of the primers were as follows:

F1: ATACACAAGTTGTGGTGCAAgagaccGAATTCggtctcAGTTTTAGA
GCTAGAAATAGC;
R1: ATGGGTTAGTTGTGGCAAAAAAAGCACCGACTCGGTGCCAC.

With the above pENTR4-gRNA7 vector as a template, the above F1 and R1 primers were used to amplify the sgRNA scaffold part, two BsaI restriction sites were added at upstream of the scaffold, for later insertion of the spacer part through the BsaI site, and 7 thymin nucleotides (T) were added to the downstream of the scaffold in the meanwhile, both ends of the amplified product also contained a sequence homologous to the Barley stripe mosaic virus vector for homologous recombination cloning, respectively, and the product was called the sgRNA expression scaffold.

4. Primers F2 and R2 were designed and synthesized by Invitrogen Company. The sequences of the primers were as follows:

F2: GCCACAACTAACCCATCTCC;
R2: CCACAACTTGTGTATCCCATTG.

With the pCB301-BSMVβ as a template, the primers F2 and R2 were used to perform inverse PCR to linearize the pCB301-BSMVβ, the 74-393 nucleotide sequence of the CP open reading frame was deleted, and the obtained product was called β-CP_{Δ74-393}.

5. The sgRNA expression scaffold obtained by PCR amplification and the linearized β-CP_{Δ74-393} were subjected for recombination reaction with the 2× Master Assembly Mix from Taihe Biotechnology Company. In the obtained product, the 74-393 nucleotide sequence of the BSMV CP was substituted with the sgRNA expression scaffold together with the upstream and downstream sequences (the total length of the CP was 597 bp. Experiments had shown that the 74-435 nucleotides of the CP were deleted, without affecting the movement of the virus, but when designing this gene editing vector, it was preferable to delete the 74-393 nucleotide sequence of the CP and insert the sgRNA expression scaffold therein). The product was called β-CP-gsca.

6. Primers F3 and R3 were designed and synthesized by Invitrogen Company. The sequences of the primers were as follows:

F3: ATGGGATACACAAGTTGTGGGGTGCTTGATGCTTTGGATAAG;
R3: ccGAATTCggtctcTTGCAACCACAGTAAGTACTTGTAGTTAAG.

With the pCB301-BSMVγ as a template, the primers F3 and R3 were used to amplify a fragment with a length of 316 bp. This fragment contains a 277 bp subgenomic promoter of the RNAγ subgenome (the γb protein was translated from the subgenomic RNA of RNAγ. The 277 bp subgenomic promoter amplified by us actually covered the core promoter of the sgRNAγ, and a certain length of extension was made at both upstream and downstream thereof. Therefore, the length of this sequence of 277 bp was not absolute), and the product was called sgγP277.

7. Primers F4 and R4 were designed and synthesized by Invitrogen Company. The sequences of the primers were as follows:

F4: TGCAAGAGACCGAATTCGGTC;
R4: CCACAACTTGTGTATCCCATTG.

With the β-CP-gsca as a template, the above primers were used to perform inverse PCR to linearize the β-CP-gsca, and the product was called linearized β-CP-gsca.

8. The above-mentioned sgγP277 and the above-mentioned linearized β-CP-gsca were subjected to recombination reaction with the 2× Master Assembly Mix from Taihe Biotechnology Company, so as to clone the sgγP277 into β-CP-gsca, and the obtained product was called β-CP-gcas.

9. Primers F5 and R5 were designed and synthesized by Invitrogen Company. The sequences of the primers were as follows:

F5: AGAGACCGAATTCGGTCTCAG;
R5: ACATCAGGACCTAGAGTTCACC.

With the above β-CP-gcas as a template, the above F5 and R5 primers were used to perform inverse PCR, to delete a sequence with a length of 85 bp from the 3′ end of the sgγP277. After treating with a T4 Polynucleotide Kinase (T4 PNK) and ligating with a T4 ligase, a product called β-CP-Tgcas was obtained.

10. Primers F6 and R6 were designed and synthesized by Invitrogen Company. The sequences of the primers were as follows:

F6: GACTCCATGGTTTTAGAGCTAGAAATAGCAAG;
R6: GCTACTACCAAACATCAGGACCTAGAGTTC.

With the above β-CP-Tgcas as a template, the above primers were used to perform inverse PCR, and self-ligation was performed after treating with T4 PNK provided by NEB Company, so as to cyclize the PCR product. The product was called BSMV β-CP-Tgcas-gNbPDS4 (also referred to as β-CP-Tgcas-gNbPDS4).

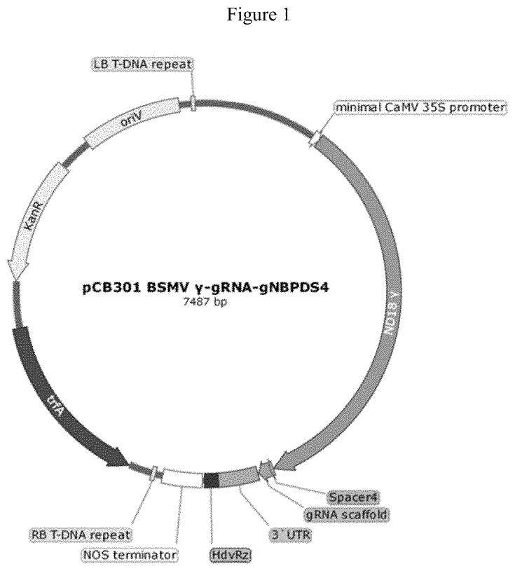

The BSMV β-CP-Tgcas-gNbPDS4 already contained a spacer of 20 bp, wherein a NcoI restriction site was contained, thus inserting a complete sgRNA sequence and a subgenomic promoter of the subgenomic RNAγ with a length of 277 bp into the pCB301-BSMVβ. The structure of the obtained BSMV VMGE vector is shown in FIG. 1.

The BSMV β-CP-Tgcas-gNbPDS4 was designed for the PDS (phytoene desaturase) gene of Nicotiana benthamiana, and therefore, the 20 bp spacer was homologous to the PDS gene. The spacer itself can be substituted as needed, and its length can also be adjusted. In addition, other methods can also be used to obtain the same cloned product.

II. Application

1. Preparation of experimental reagents for Agrobacterium-mediated transient expression

For reagents preparation and inoculation methods, please refer to Li Zhenggang's doctoral dissertation (Functional analysis of Barley stripe mosaic virus TGB1 protein in nuclear-cytoplasmic trafficking and hijacking of the nucleolar protein fibrillarin, Li Zhenggang, Doctoral Dissertation, China Agricultural University, 2017).

The reagents used include: 1 M MES (Morpholineethanesulfonic acid), 50 mM As (Acetosyringone) and 1 M MgCl₂. They were diluted to a final concentration of 10 mM MES, 10 mM MgCl₂ and 150 μM As to obtain the Infiltration Buffer.

2. Agrobacterium-mediated infiltration (Agroinfiltration) method was used to inoculate Nicotiana benthamiana plants:

The pCB301-BSMVα, BSMVβ-CP-Tgcas-gNbPDS4, pCB301-BSMVγ and pHSE401 were transformed into Agrobacterium strain EHA105, respectively. Single colonies were picked and grown overnight in the LB liquid medium harboring antibiotics at 28° C. with constant shaking. Centrifuge was performed at 4000 rpm for 10 min, the supernatant was discarded, and the Infiltration Buffer was used to resuspend the cells in the pellet. The OD₆₀₀ of the suspension was measured with a UV spectrophotometer, and adjusted to OD₆₀₀ of 0.3 for the BSMV VMGE vector harboring Agrobacterium and OD₆₀₀ of 0.5 for the Agrobacterium transformed with pHSE401, using the Infiltration Buffer and mix well. The Agrobacterium mixture was incubated in a 28° C. incubator for 2 to 4 h, and infiltrated into the Nicotiana benthamiana leaves of 4 to 6 weeks old with a sterilized needleless syringe.

### Example 2

In this Example, the Nicotiana benthamiana PDS gene was used as the target gene to illustrate the construction and application of a BSMV-based gene editing vector system.

I. Construction

The templates involved in the following steps were related to Example 1. The specific steps were as follows:

1. Primers F7 and R7 were designed and synthesized by Invitrogen Company. The sequences of the primers were as follows:

F7: AAAAAAAAAAAAATGTTTGATCAGATCATTCAAATCTGATGGTGCCC
ATC;
R7: TTACTTAGAAACGGAAGAAGAATCATCACATCCAACAGAAT.

With the above-mentioned pCB301-BSMVγ as a template, the primers F7 and R7 were used to linearize the pCB301-BSMVγ by inverse PCR, and the obtained product was called linearized pCB301-BSMVγ.

2. Primers F8 and R8 were designed and synthesized by Invitrogen Company. The sequences of the primers were as follows:

F8: TCTTCTTCCGTTTCTAAGTAAGGTGCTTGATGCTTTGGATAAGGC;
R8: GAATGATCTGATCAAACATTTTTTTTTTTTTAAAAAAAGCACCGACT
CGGTGCC.

With the above-mentioned β-CP-gcas as a template, the primers F8 and R8 were used to perform PCR, the amplified product contained a complete sgRNA scaffold and also contained two oppositely arranged BsaI restriction sites. The obtained product was called sgRNA-cas.

3. The above-mentioned linearized pCB301-BSMVγ and the sgRNA-cas were subjected to recombination reaction with the 2× Master Assembly Mix from Taihe Biotechnology Company, and the obtained product was called γ-gcas.

4. Primers F9 and R9 were designed and synthesized by Invitrogen Company. The sequences of the primers were as follows:

F9: GACTCCATGGTTTTAGAGCTAGAAATAGCAAGTTAAAATAAGGC;
R9: GCTACTACCAATTACTTAGAAACGGAAGAAGAATCATCACATC.

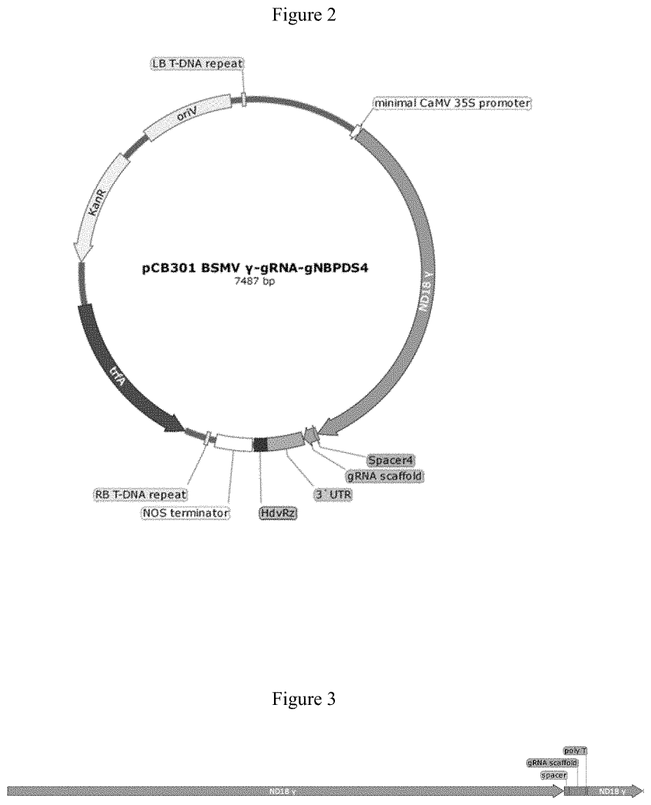

After the primers F9 and R9 were used to perform inverse PCR with the above-mentioned γ-gcas as a template, the product was subjected to treatment with a T4 PNK enzyme and then ligated with T4 ligase, after which the above-mentioned sgγP277 sequence was removed while the complete sgRNA sequence was maintained in the product. The 20 bp spacer contained in the obtained product was homologous to the Nicotiana benthamiana PDS gene. The obtained product was called pCB301-BSMV γ-gRNA-gNbPDS4 (also referred to as γ-gRNA-gNbPDS4). The structure of the obtained gene editing vector was shown in FIG. 2.

For other gene targets, F9 and R9 can be designed according to needs to substitute the spacer with a required sequence, so as to be used to edit different genes and different targets.

II. Application

Agrobacterium-mediated infiltration method was used to inoculate Nicotiana benthamiana:

The pCB301-BSMVα, pCB301-BSMVβ and pCB301-BSMVγ-gRNA-gNbPDS4 were transformed into Agrobacterium strain EHA105. Single colonies were picked and grown overnight in the LB liquid medium harboring antibiotics at 28° C. with constant shaking. Centrifuge was performed at 4,000 rpm for 10 min, the supernatant was discarded, and the Infiltration Buffer was used to resuspend the cells in the pellet. The OD₆₀₀ of the suspension was measured with a UV spectrophotometer, and adjusted to OD₆₀₀ of 0.3 for the Agrobacterium harboring BSMV VMGE vector and OD₆₀₀ of 0.5 for the Agrobacterium containing with pHSE401 using the Infiltration Buffer and mix well. The Agrobacterium mixture was incubated at room temperature for 2 to 4 h followed by infiltration into the Nicotiana benthamiana leaves of 4 to 6 weeks old with a sterilized needleless syringe.

### Experimental Example 1

This Experimental Example is used to illustrate the editing effect of Example 1 (due to the integration of sgRNA in the vector containing the RNAβ, using the characteristic vector β-CP-Tgcas-gNbPDS4 in the Figures and hereinafter of Example 1 to represents the complete vector system, i.e. the pCB301-BSMVα, BSMVβ-CP-Tgcas-gNbPDS4, pCB301-BSMVγ) and Example 2 (due to the integration of sgRNA in the vector containing the RNAγ, using the characteristic vector γ-gRNA-gNbPDS4 in the Figures and hereinafter of Example 2 to represents the complete vector system, i.e. the pCB301-BSMVα, pCB301-BSMVβ and pCB301-BSMVγ-gRNA-gNbPDS4) to the target gene (Nicotiana benthamiana PDS gene).

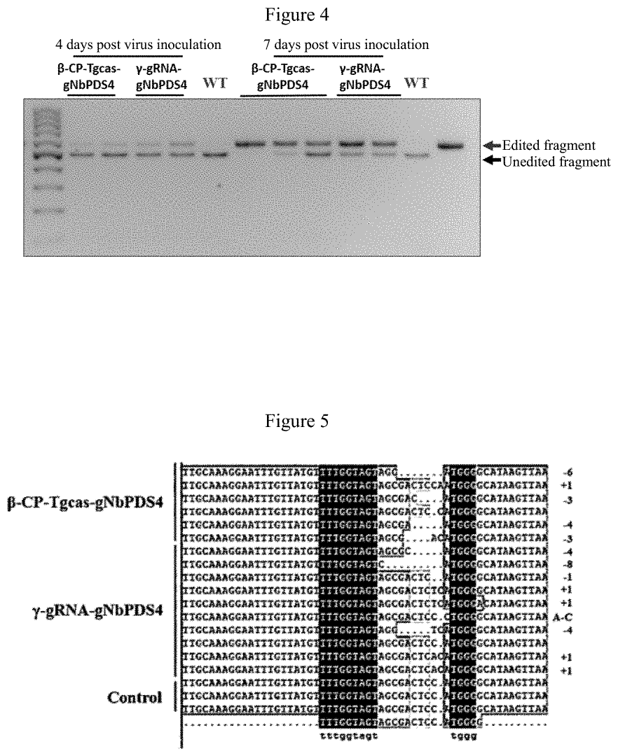

The BSMV gene editing vector was inoculated by Agrobacterium-mediated infiltration and the Cas9 protein was also transiently expressed by Agrobacterium-mediated infiltration, with a concentration of the OD₆₀₀=0.3 for β-CP-Tgcas-gNbPDS4 or γ-gRNA-gNbPDS4 vectors, and a concentration of the OD₆₀₀=0.5 for the Cas9 protein expression vector pHSE401. At 4 and 7 days post inoculation (dpi), genomic DNA from the inoculated leaves was extracted with the CTAB method, and the extracted genomic DNA was used as a template to amplify a 544 bp DNA fragment that containing the target site, which harboring a NcoI restriction site, the product was called NbPDS544. After NcoI digestion, results of gel-electrophoresis showed that, compared with the NbPDS544 product amplified from the DNA extracted from healthy leaves, the digested products showed that a NcoI-resistant band was present in the samples for both the ge-BSMVβ-gNbPDS and the ge-BSMVγ-gNbPDS-infiltrated leaves, suggesting that mutations occurred at the target sites (as shown in FIG. 4).

The NcoI-digested NbPDS544 was then ligated into a T-vector for sequencing. It can be seen that different types of mutations occurred at the target sites, including base insertions, base deletions, and base substitutions, which confirmed that targeted gene editing indeed occurred at the target sites, and these mutations were not present in the control group (as shown in FIG. 5).

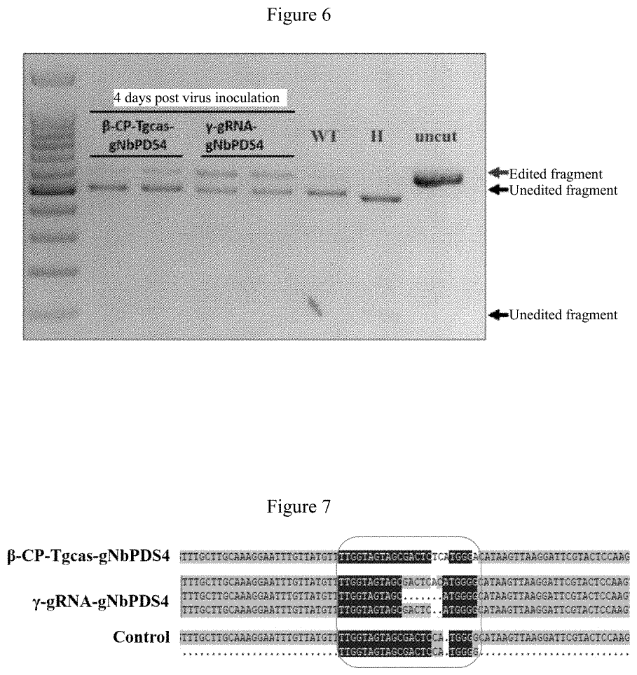

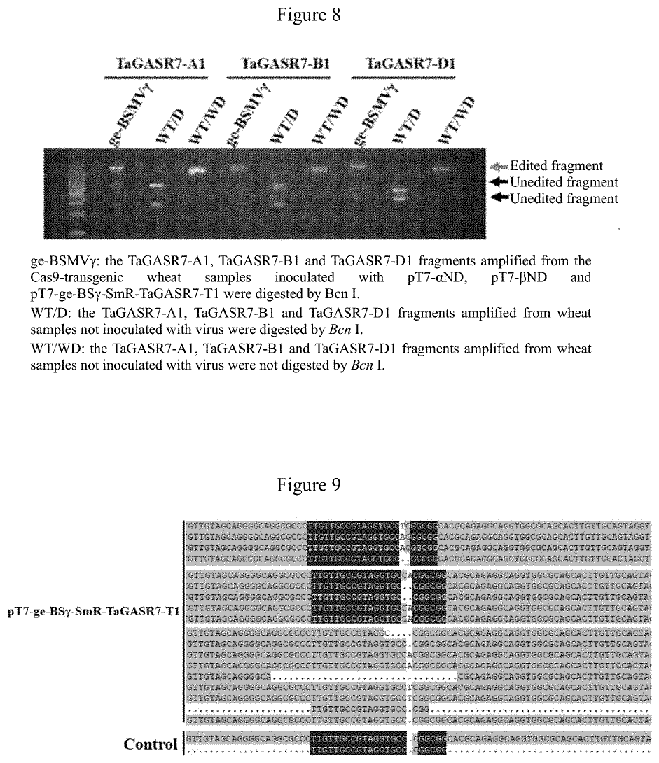

The gene editing results of β-CP-Tgcas-gNbPDS4 and γ-gRNA-gNbPDS4 on the target gene (Nicotiana benthamiana PDS gene) in the systemic leaf are shown in FIG. 8. Nicotiana benthamiana was inoculated with the wild-type BSMV or the BSMV-based VMGE vectors containing β-CP-Tgcas-gNbPDS4 and γ-gRNA-gNbPDS4, respectively. After systemic infection, the Cas9 protein was transiently expressed in the systemic leaves via Agrobacterium-mediated infiltration. Three days later, genomic DNA of the infected systemic leaves transiently expressing the Cas9 protein was extracted by the CTAB method, and the extracted genomic DNA was used as a template to amplify a 544 bp DNA fragment that containing the target site, which harboring a NcoI restriction site, and the product was called NbPDS544. After NcoI digestion, results of gel-electrophoresis showed that, compared with the NbPDS544 product amplified from the DNA templated that extracted from the healthy leaves and the wild type BSMV-infected leaves, the digested products showed that a NcoI-resistant band was present in the samples for both the ge-BSMVβ-gNbPDS and the ge-BSMVγ-gNbPDS-infected systemic leaves, suggesting that targeted gene editing occurred at the target sites (as shown in FIG. 6).

The NbPDS544 fragment amplified from the systemic leaves was then ligated into a T-vector for sequencing. It can be seen that different types of mutations occurred at the target sites, including base insertions, base deletions, and base substitutions, which confirmed that targeted gene editing indeed occurred at the target sites, and these mutations were not present in the control groups (as shown in FIG. 7).

### Example 3

In this example, wheat TaGASR7 gene and maize ZmTMS5 gene were used as target genes to illustrate the construction of a BSMV-based VMGE vector system and its application in wheat and maize.

I. Construction

To inoculate wheat and maize, in vitro transcripts of the BSMV VMGE vectors were used to rub-inoculate the of the leaves. The basic vectors involved include pT7-α_{ND}, pT7-β_{ND}, and pT7-γ_{ND} (Petty, I. T. D., Hunter, B. G., Wei, N. & Jackson, A. O. (1989). Infectious Barley stripe mosaic virus RNA transcribed in vitro from full-length genomic cDNA clones. Virology, 171, 342-349) provided by Professor Andrew O. Jackson and the pT7-β_{G404E} vector which is obtained by mutating amino acid G (glycine) at position 404 of TGB1 in pT7-β_{ND} into amino acid E (glutamic acid).

1. Primers F10 and R10 were designed and synthesized by Invitrogen Company. The sequences of the primers were as follows:

F10: GTTTTAGAGCTAGAAATAGCAAGTTAAAATAAGGC;
R10: TTACTTAGAAACGGAAGAAGAATCATCACATCC.

With the above-mentioned pCB301-BSMVγ-gRNA-gNbPDS4 as a template and F10 and R10 as primers, high-fidelity PCR amplification was performed, and the obtained product was called linearized pCB301-ge-BSγ.

2. A double-stranded DNA fragment called SmR861 with a length of 861 bp was synthesized. This fragment contains two Sap I restriction sites arranged in opposite directions. The 5′→3′ sequence of this DNA fragment is as follows:

GGATGTGATGATTCTTCTTCCGTTTCTAAGTAACGAAGAGCatgggggaag
cggtgatcgccgaagtatcgactcaactatcagaggtagttggcgtcatcg
agcgccatctcgaaccgacgttgctggccgtacatttgtacggctccgcag
tggatggcggcctgaagccacacagtgatattgatttgctggttacggtga
ccgtaaggcttgatgaaacaacgcggcgagctttgatcaacgaccttttgg
aaacttcggcttcccctggagagagcgagattctccgcgctgtagaagtca
ccattgttgtgcacgacgacatcattccgtggcgttatccagctaagcgcg
aactgcaatttggagaatggcagcgcaatgacattcttgcaggtatcttcg
agccagccacgatcgacattgatctggctatcttgctgacaaaagcaagag
aacatagcgttgccttggtaggtccagcggcggaggaactctttgatccgg
ttcctgaacaggatctatttgaggcgctaaatgaaaccttaacgctatgga
actcgccgcccgactgggctggcgatgagcgaaatgtagtgcttacgttgt
cccgcatttggtacagcgcagtaaccggcaaaatcgcgccgaaggatgtcg
ctgccgactgggcaatggagcgcctgccggcccagtatcagcccgtcatac
ttgaagctagacaggcttatcttggacaagaagaagatcgcttggcctcgc
gcgcagatcagttggaagaatttgtccactacgtgaaaggcgagatcacca
aggtagtcggcaaataaGCTCTTCGGTTTTAGAGCTAGAAATAGC.

3. The linearized pCB301-ge-BSMVγ and the SmR861 were subjected to recombination reaction with the 2× Master Assembly Mix from Taihe Biotechnology Company, and the obtained product was called pCB301-ge-BSγ-SmR.

4. Primers F11 and R11 were designed and synthesized by Invitrogen Company. The sequences of the primers were as follows:

F11: AAAAAAAAAAAAATGTTTGATCAGATCATTCAAATCTGATGGTGCC
CATC;
R11 (i.e., R7): TTACTTAGAAACGGAAGAAGAATCATCACATCCAA
CAGAAT.

With the above-mentioned pT7-γ_{ND} as a template and F11 and R11 as primers, high-fidelity PCR amplification was performed, and the obtained product was called linearized pT7-γ_{ND}.

5. Primers F12 and R12 were designed and synthesized by Invitrogen Company. The sequences of the primers were as follows:

F12: TTCTTCTTCCGTTTCTAAGTAACGAAGAGCatgggggaagcggtga
t;
R12 (i.e.. R8): GAATGATCTGATCAAACATTTTTTTTTTTTTAAAA
AAAGCACCGACTCGGTGCC.

With the above-mentioned pCB301-ge-BSγ-SmR as a template and F12 and R12 as primers, high-fidelity PCR amplification was performed, and the obtained product was called ge-BSγ-SmR.

6. The above-mentioned linearized pT7-γ_{ND} and the ge-BSγ-SmR were subjected to recombination reaction with the 2× Master Assembly Mix from Taihe Biotechnology Company, and the obtained product was called pT7-ge-BSγ-SmR.

7. Primers F15 and R15 were designed and synthesized by Invitrogen Company. The sequences of the primers were as follows:

F15: TAATTGTTGCCGTAGGTGCCCGG;
R15: AACCCGGGCACCTACGGCAACAA.

8. F15 and R15 were diluted to a concentration of 100 μM, respectively. F15 and R15 were treated with T4 PNK, and the 50 μL reaction mix was as follows: F15 20 μL, R15 20 μL, 10×T4 ligase buffer (NEB) 5 μL, ddH₂O 4 μL, T4 PNK (NEB) 1 μL. Reaction was performed in a 37° C. incubator for 45 min.

9. The reaction mix was then transferred into a PCR tube, and double strand annealing was performed in a PCR instrument to allow spontaneously base-pairing of F15 and R15 to form a double-stranded DNA fragment. The reaction conditions were as follows:

Denaturation at 95° C. for 5 min and gradually chilled to 25° C. with 1° C. drops per minute. A final step was performed by maintaining the samples at 16° C. for 10 min. After the reaction was completed, the product was taken out in time and placed on ice for use in the subsequent reaction or stored at −20° C. The product was called Oligo-TaGASR7-T1.

10. The pT7-ge-BSγ-SmR was digested with the SapI restriction endonuclease produced by NEB with a final concentration of 20 ng/μL. The product was called SapI linearized pT7-ge-BSγ-SmR.

11. Ligation. The T4 ligase produced by NEB was used to ligate the Sap I linearized pT7-ge-BSγ-SmR and Oligo-TaGASR7-T1. The 20 μL reaction mix was as follows: Oligo-TaGASR7-T1 10 μL, 10×T4 ligase buffer (NEB) 2 μL, SapI-linearized pT7-ge-BSγ-SmR 1 μL, ddH₂O 6 μL, T4 ligase (NEB) 1 μL. Ligation was performed at room temperature (about 20° C.) for 2 h or more, or ligation was performed at 16° C. overnight.

12. The ligation product was transformed into Escherichia coli strain JM109. After culturing, positive colonies were screened to extract the plasmid and sequencing was performed. The correct clone was screened, called pT7-ge-BSγ-SmR-TaGASR7-T1.

13. To construct the pT7-ge-BSγ-SmR-ZmTMS5-T2, the F15 and R15 were substituted with the follows:

F17: TAAGGTGAAGCAGAAGCTTAAGC;
R17: AACGCTAAGCTTCTGCTTCACC.

Then go back to step 9 to step 12 to obtained the pT7-ge-BSγ-SmR-ZmTMS5-T2.

### Experimental Example 2

The present experimental example was used to illustrate the editing effect of Example 3 on the target genes (wheat TaGASR7 gene and maize ZmTMS5 gene).

Inoculating the wheat and maize with in vitro transcripts of the BSMV vectors mentioned above. The wheat line used was provided by Researcher Sientist Xia Lanqin from Institute of Crop Science, Chinese Academy of Agricultural Sciences, and the maize used was provided by Dr. Zhao Haiming from China Agricultural University. The wheat and maize lines used were transformed to express the Cas9 protein. For in vitro transcription, pT7-ge-BSγ-SmR-TaGASR7-T1, pT7-ge-BSγ-SmR-ZmTMS5-T2 and pT7-α_{ND} were linearized with MluI. While pT7-β_{ND} and pT7-β_{G404E} were linearized with SpeI. 200-400 ng of linearized plasmids were used as template for transcription, and the following reagents were added: 6 μL 5× Trans Buffer, 3 μL 100 mM DTT, 30 U (unit) HPRI, 2 μL rNTP (ATP, UTP and CTP were each 10 mM, GTP was 1 mM) (Shanghai Sangon Biotech), 10 U T7 RNA polymerase (Promega), 5 mM Ribo m⁷G Cap Analog (Promega), and DEPC treated ddH₂O was added to a final volume of 30 μl. 2 μl products were taken for electrophoresis analysis for quality control after 3 to 5 h reaction at 37° C. in an incubator. The remaining in vitro transcripts were mixed according to the ratio of 1:1:1 (for RNAα, RNAβ and RNAγ of the BSMV VMGE vectors), 2×FES buffer of the same volume was added and mixed with the in vitro transcripts to mechanically inoculate the leaves of wheat of and maize that grown to the two-leaf stage.

At 14- and 30-days post inoculation, leaves were collected, respectively, and the genomic DNA was extracted for mutation analysis.

Analysis Methods:

The extracted genomic DNA from wheat and maize leaves were used as PCR templates. For wheat, the following primers were used: primer F16: CCTTCATCCTTCAGCCATGCAT, paired with primer R16-A: CCACTAAATGCCTATCACATACG, primer R16-B: AGGGCAATTCACATGCCACTGAT and primer R16-D: CCTCCATTTTTCCACATCTTAGTCC, respectively.

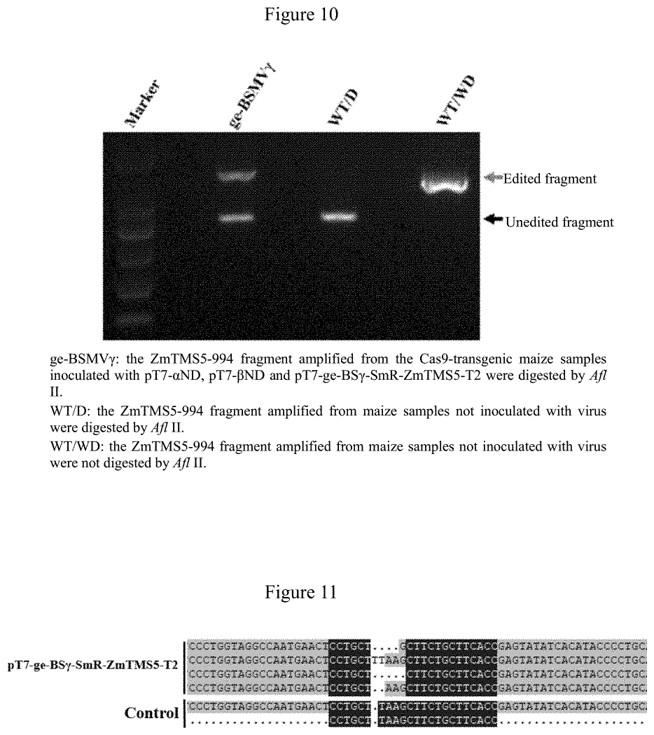

High-fidelity PCR amplification was performed to obtain the target sites-containing DNA fragments with lengths of 560 bp, 569 bp, and 582 bp, respectively, called TaGASR7-A1, TaGASR7-B1, and TaGASR7-D1. The target sites of these three fragments all contained a BcnI restriction site. After BcnI digestion, gel-electrophoresis showed that, compared with the TaGASR7-A1, TaGASR7-B1 and TaGASR7-D1 products amplified from the DNA template extracted from healthy leaves, the digested products showed that a BcnI-resistant band was present in the samples from both the ge-BSMVβ-gNbPDS and the pT7-ge-BSγ-SmR-TaGASR7-T1-inoculated leaves, suggesting that mutations occur at the target sites (as shown in FIG. 8). The sequencing results for the TaGASR7-A1, TaGASR7-B1, and TaGASR7-D1 that cloned into the T-vector are shown in FIG. 9. For maize, high-fidelity PCR amplification was performed with primers F18: TCAAGAGACTTGCGTCATCTTCCC and R18: GCATGCTCAACTGAAATTGAGTCGTC to obtain a target site-containing DNA fragment with a length of 994 bp, i.e., ZmTMS5-994. The target site of this fragment contained an Afl II restriction site. After AflII digestion, gel-electrophoresis showed that, compared with the ZmTMS5-994 product amplified from the DNA template extracted from healthy leaves, the digested products showed that a AflII-resistant band was present in the samples from the pT7-ge-BSγ-SmR-ZmTMS5-T2-inoculated leaves, suggesting that targeted mutations occur at the target sites (as shown in FIG. 10).

The AflII-digested ZmTMS5-994 was then cloned into a T-vector for sequencing. It can be seen that different types of mutations occurred at the target sites, including some base insertions, base deletions, and base substitutions, which confirmed that targeted mutations indeed occurred at the target sites, and these mutations were not present in the control (as shown in FIG. 11).

### Experimental Example 3

The present Experimental Example was used to illustrate the editing effect of the BSMV gene editing vector on wheat pollen, and the following effect can be obtained: wheat seeds containing edited target gene can be obtained by inoculation and the edited target gene can be transmitted to progeny wheat plants.

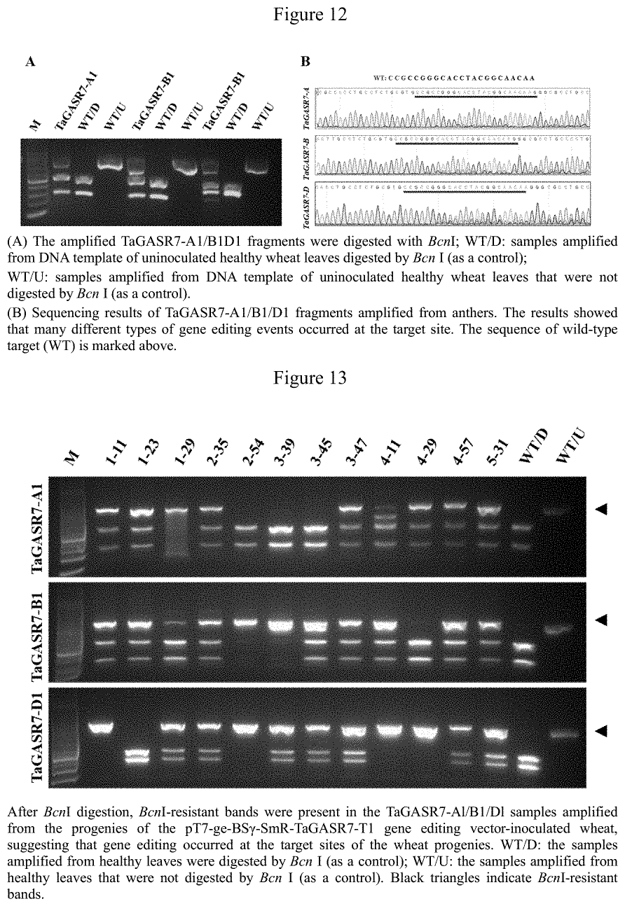

With the same inoculation method as in Experimental Example 2, the pT7-α_{ND}, pT7-β_{ND} and pT7-ge-BSγ-SmR-TaGASR7-T1 described in Experimental Example 2 were used to inoculate Cas9 transgenic wheat. The Cas9 transgenic wheat can be inoculated at the vegetative growth stage or the reproductive growth stage, which can be before or after the heading stage of wheat. Preferably, the wheat in the vegetative growth stage was used for inoculation in the present Experimental Example. The BSMV gene editing vector system was inoculated, after systemic infection and flowering of the inoculated wheat, anthers were collected to extract genomic DNA, and the effect of gene editing was tested by BcnI digestion and sequencing. Refer to Experimental Example 2 for the method. In brief, after extracting genomic DNA from the anthers, primer F16: CCTTCATCCTTCAGCCATGCAT were used in combination with primers R16-A: CCACTAAATGCCTATCACATACG, R16-B: AGGGCAATTCACA TGCCACTGAT, and R16-D: CCTCCATTTTTCCACATCTTAGTCC, respectively, to amplify DNA fragments of 560 bp, 569 bp, and 582 bp in length containing the target sites (called TaGASR7-A1, TaGASR7-B1, and TaGASR7-D1, respectively) from three subgenomes of wheat by high-fidelity PCR. The target sites of these three fragments all contained a BcnI restriction site. After BcnI digestion, gel-electrophoresis showed that, compared with the TaGASR7-A1, TaGASR7-B1 and TaGASR7-D1 products amplified from the DNA template extracted from uninoculated healthy wheat leaves, the digested products showed that a BcnI-resistant band was present in the samples from the pT7-ge-BSγ-SmR-TaGASR7-T1-inoculated leaves, suggesting that mutations occurred at the target sites (as shown in FIG. 12A). The amplified TaGASR7-A1, TaGASR7-B1, and TaGASR7-D1 were subjected to sequencing, and the results further proved that different types of mutations occurred in the target genes (as shown in FIG. 12B), which proved that the BSMV-based gene editing vector system can directly perform gene editing on wheat anthers in vivo.

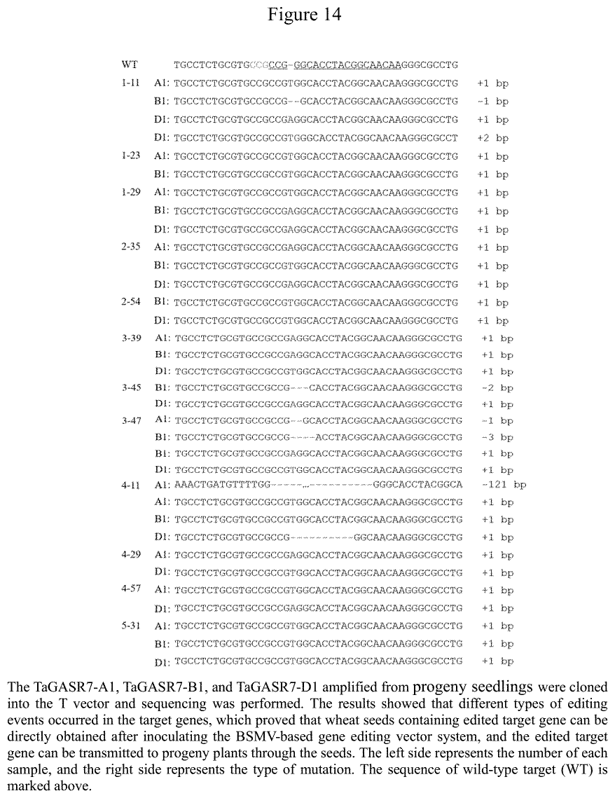

Furthermore, the seeds of the Cas9 transgenic wheat inoculated with pT7-α_{ND}, pT7-β_{ND} and pT7-ge-BSγ-SmR-TaGASR7-T1 were harvested, and after the seeds were sown, the genomic DNA of the grown progeny seedlings was extracted. With the same manner as above, the target sites-containing DNA fragments with lengths of 560 bp, 569 bp, and 582 bp (called TaGASR7-A1, TaGASR7-B1, and TaGASR7-D1, respectively) were amplified from three subgenomes of wheat progenies by high-fidelity PCR. The target sites of these three fragments all contained a BcnI restriction site. After BcnI digestion, gel-electrophoresis showed that, compared with the TaGASR7-A1, TaGASR7-B1 and TaGASR7-D1 products amplified from the DNA template extracted from uninoculated healthy wheat leaves, the digested products showed that a BcnI-resistant band was present in the genomic DNA extracted from the pT7-ge-BSγ-SmR-TaGASR7-T1-inoculated progeny seedlings leaves, suggesting that mutations occurred at the target sites (as shown in FIG. 13). The amplified TaGASR7-A1, TaGASR7-B1, and TaGASR7-D1 were cloned into the T vector and sequencing was performed. The results further proved that different types of editing occurred in the target genes (as shown in FIG. 14), which proved that wheat seeds containing edited target gene can be directly obtained after inoculating with the BSMV-based gene editing vector system, and the edited target gene can be transmitted to progeny plants through the seeds, without the need for transformation, tissue culture and regeneration process.

It should be understood that, after the number of reagents or raw materials used in the above examples is expanded or reduced in equal proportion, the technical solutions are substantially the same as those of the above examples.

Although the general description and specific embodiments have been used to describe the present invention in detail above, it is obvious to those skilled in the art that some modifications or improvements can be made on the basis of the present invention. Therefore, these modifications or improvements made without departing from the spirit of the present invention fall within the protection scope of the present invention.

### INDUSTRIAL APPLICABILITY

The present invention provides a Barley stripe mosaic virus-based gene editing vector system. The gene editing vector system comprises artificial plasmids separately containing Barley stripe mosaic virus RNAα, RNAβ, and RNAγ. The required sgRNA sequence is integrated in RNAβ or RNAγ. The Barley stripe mosaic virus-based gene editing vector system can achieve efficient gene editing on genomes of dicotyledons such as Nicotiana benthamiana and monocotyledons such as wheat and maize, and can directly obtain wheat seeds containing edited target gene by inoculating the Cas9-transgenic wheat, and the gene editing events can be transmitted to progeny plants through the seeds, without the need for transformation, tissue culture and regeneration processer. Thus, the present application shows good application prospects for basic research and crop improvement.

What is claimed is:

1. A Barley stripe mosaic virus-based gene editing vector system, comprising artificial plasmids separately containing Barley stripe mosaic virus RNAα, RNAβ, and RNAγ, wherein a required sgRNA sequence is integrated into RNAβ or RNAγ.

2. The gene editing vector system according to claim 1, wherein the required sgRNA sequence is integrated at 5′ end or 3′ end of γb in RNAγ or at a middle part of the coding sequence of coat protein CP in RNAβ.

3. The gene editing vector system according to claim 2, wherein sgRNA expression scaffold together with upstream and downstream sequences are subjected to insertion or substitution in the region between 74 bp and 435 bp of the coat protein CP coding sequence.

4. The gene editing vector system according to claim 1, wherein the artificial plasmids contain a HDVRz ribozyme.

5. The gene editing vector system according to claim 4, wherein the artificial plasmids include, but are not limited to pCB301 or pCass4-Rz.

6. The gene editing vector system according to claim 1, wherein, the gene editing vector system can directly obtain wheat seeds harboring targeted gene editing events by inoculating the Cas9-transgenic wheat, and the edited target gene is transmitted to progeny plants through the seeds, without the need for transformation, tissue culture and regeneration process.

7. A method for gene editing of plants, wherein the gene editing vector system according to claim 1 is used for gene editing.

8. The method according to claim 7, wherein the plants are monocotyledons or dicotyledons.
